(19) United States
(12) Patent Application Publication
(10) Pub. No.: US 2023/0172590 A1
(43) Pub. Date: Jun. 8, 2023

(54) SINGLE-USE SAMPLING DEVICE CONFIGURED TO COLLECT, TRANSPORT AND DISPENSE ORAL FLUID SAMPLES TO MULTIPLE TESTING SITES

(71) Applicant: James Ramsey, Norfolk, VA (US)

(72) Inventor: James Ramsey, Norfolk, VA (US)

(21) Appl. No.: 18/062,052

(22) Filed: Dec. 6, 2022

Related U.S. Application Data
(60) Provisional application No. 63/286,645, filed on Dec. 7, 2021.

(51) Int. Cl. A61B 10/00; B01L 3/00

(52) U.S. Cl. CPC A61B 10/0051; B01L 3/5029; B01L 3/5023; B01L 2300/069

(57) ABSTRACT

An oral sample collection system configured to draw and deliver an oral sample to a first analyte testing device (such as a local dry reagent test), and then allow the same sample to then be retested at a remote laboratory. The system uses a swab holder with a hollow tubular handle, an outer swab mounted at one end of the handle, and an inner swab mounted on a carriage positioned inside the hollow handle. Sample is collected in both swabs, the outer swab sample used for immediate testing, and the inner swab sample retained for later retesting as needed. This device may be used with a collection container configured to accept the swab holder and conduct immediate tests on the outer swab sample. Various applications, including utility for drugs of abuse testing, are also discussed.

### CROSS REFERENCE TO RELATED APPLICATIONS

This application claims the priority benefit of U.S. provisional patent application 63/286,645, filed Dec. 7, 2021, the entire contents of which are incorporated herein by reference.

### BACKGROUND OF THE INVENTION

### Field of the Invention

This invention is in the field of laboratory testing and oral sample collection devices.

### Description of the Related Art

Oral fluid sampling devices: Simple, easy to use, oral sampling devices configured to obtain a Buccal cavity oral fluid (saliva) sample, and deliver this saliva sample to a laboratory instrument for subsequent testing, are known in the art. These prior art devices include the OraSure oral specimen collection device, produced by Orasure Technologies, Inc., Bethlehem Pa. These devices are configured to be used by non-skilled users. These devices are often used for clinical medical testing, drugs of abuse testing, and other testing purposes.

Such devices typically operate by providing a bibulous fluid absorbing pad, often affixed at the end of a plastic stick, and an optional storage container with an optional preservative solution which may contain various preservatives. Patients or other human users will typically use the stick to place the pad into the patient's mouth (often between the gums and cheek). Over a few minutes, the pad will absorb saliva. The pad and stick are then removed and may be placed in an optional storage container with an optional preservative solution. The saliva in the pad may then be placed in a testing device, and various analytes in the saliva then tested. If this is done immediately, no transport container or preservative may be needed. Alternatively, if the sample is to be tested later, or transported and then tested later at a remote testing site, the pad and stick may be inserted into the optional storage container, optionally further preserved by the optional preservative solution, and then tested later.

Analyte testing methodologies: A previous commonly owned and invented patent application Ser. No. 16/038,512 (US patent publication 2019/0057759), published Feb. 21, 2019, now U.S. Pat. No. 10,340,032 published Jul. 2, 2019), the entire contents of which are incorporated herein by reference, described a rapidly reconfigurable drug detection system with enhanced confidentiality.

This invention described a point of care drug of abuse test system configured to allow a small number of general test kits to be used to analyze many different combinations of analytes while preserving privacy and the chain of custody. The system used disposable multiple-analyte test kits and allows the operator to select which subset of kit analytes to run. The computerized device images the test kits and transmits test results to a remote server along with a test-specific ID code.

### BRIEF SUMMARY OF THE INVENTION

In some embodiments, it is useful to analyze various analytes, such as various drugs of abuse, more than once. For example, after an initial round of testing using a locally situated disposable test kit, it may be desired to run at least some samples, such as samples initially identified as being problematic, at other test facilities to further verify at least some samples initially identified as being problematic. Additionally, for quality control purposes, it may also be desirable to run the same sample using locally situated test kits, as well as various remote reference laboratories.

One problem with prior art sampling methods, such as oral (saliva) based sampling methods, is that such prior art methods are primarily oriented towards single-use applications. However, for purposes such as described above, where it may be desired to run the same sample twice, both locally on a dry reagent test kit, and then remotely at a validated reference laboratory. In such applications, it can be critical to configure the system in a way that, to adequate legal standards, preserves the chain of evidence and ensures adequate tracking and confidentiality.

What is needed is an improved oral sample collection system that can be used at least twice—once to draw and deliver an oral sample to a first analyte testing device (such as a local dry reagent test), and allow this same sample to then be retested at a remote laboratory. Since some applications, such as drugs of abuse testing, can have negative legal consequences on the individuals being tested, it is further particularly useful if the oral sample collection system is designed to ensure that the sample tested at a local testing facility (or laboratory A) is exactly the same sample that is tested at a remote or reference testing facility (or laboratory B).

In some embodiments, the invention may be a method of collecting oral fluids for testing. This method may comprise using an oral fluid collection system to obtain saliva from a patient. This oral fluid collection system will typically comprise at least a first swab and a second swab, where both swabs will typically comprise a bibulous hydrophilic material. According to the method, the collection system is used to collect saliva on both swabs simultaneously. Thereinafter, saliva retained on the first swab may be tested, while saliva stored on the second swab may be retained in another part of the oral fluid collection system for possible later testing.

In some embodiments, the invention comprises an oral sample collection device, system, or method. Expressing the concept in device format, the invention may comprise a handheld saliva collection system with two absorbent swabs (an outer swab and an inner swab) mounted on a hollow transparent plastic handle. The inner swab extends into the hollow plastic handle and is used both to help mount the outer swab onto the handle, as well as serve to store a portion of the saliva sample.

In use, the oral sample donor places the distal (mouth end) of the device, which contains the outer and inner swab, into the mouth and where both swabs can absorb saliva. Over the course of a few minutes, saliva enters both swabs, and gradually passes up the inner swab to the proximal (user hand side) of the hollow plastic handle. An optional first tracking dye (such as a red dye) may be placed in dry form on the proximal (user hand side) of the inner swab, so that when the saliva finally reaches the extreme proximal (user hand side) of the inner swab, the first tracking dye will start to migrate. This can be visually assessed through the transparent handle, and lets the donor (or operator or other user) know when enough saliva has been applied.

The oral sampling device can then be removed from the donor's mouth, and some saliva sample immediately expelled from the outer swab (and portions of the inner swab inside of the outer swab) by application of pressure. These can be used for on-site tests. Once this is done, the oral sampling device, which will still contain some of the saliva sample on the inner swab inside the hollow handle, can be placed (screwed into) a transport tube. This transport tube may optionally contain a preservative solution and an optional second tracking dye. This will usually be a contrasting color, so that if the first tracking dye is a red dye, the second tracking dye may be a blue dye. This allows the gradual wicking of the preservative solution up the outer swab and inner swab (towards the proximal end of the inner swab) to be visually assessed, as desired.

At the second (remote) laboratory, the portion of the inner swab remaining inside of the hollow handle can be removed, and the status of the tracking dyes used to determine if the inner swab needs to be trimmed to avoid any dilution or contamination by the preservative solution. The saliva remaining inside of the inner swab can then be expelled, usually by application of mechanical pressure, and then used for subsequent testing at the second (remote) laboratory site.

### BRIEF DESCRIPTION OF THE DRAWINGS

FIG. 1 shows a cross-sectional diagram of the oral collection device, showing the inner swab, outer swab, swab holder, and the inner swab carriage. The proximal end of the swab holder has a removable cap. The swab holder also has an optional container interface, and a hollow tubular handle. A moveable inner swab carriage holding an inner swab is positioned inside of the hollow tubular handle. The distal end of the swab holder has an outer swab support, and a cylindrical outer swab with an opening along the central axis of the outer swab is positioned on the distal end of the outer swab support. Prior to use, the movable inner swab carriage and the inner swab are disposed inside this opening, as well as inside the hollow tubular handle.

FIG. 2 shows the oral sample collection device inserted into a patient's mouth in order to obtain a saliva sample.

FIG. 3 shows a cross sectional view of both the oral collection device and the optional container, which in this case is a drug screen container.

FIG. 4 shows how after the sample donor has applied saliva to the oral sample collection device, the collection device is inserted into the container. In this process, the oral sample (saliva) permeates both the outer swab and the inner swab.

FIG. 5 shows the next step of the process. Here the collection device is pressed down into the container. This compresses the outer swab, expelling saliva from the outer swab into the bottom of container. This also forces the inner swab carriage and the inner swab further towards the proximal end of the hollow tubular handle. Note that in some embodiments, the container may be transparent, and have one or more test devices, such as lateral flow immunoassays, arranged on the inner sides of the container. These optional assays can be activated by the sample, allowing one or more rapid tests (such as drugs of abuse tests) to be quickly performed. At the same time, a portion of the original sample is preserved in the inner swab, and this can optionally be tested at a later date. To do this, the container and oral collection device assembly may be preserved intact for further use.

FIG. 6 shows how the duplicate sample preserved in the inner swab may be accessed. Here the removable cap may be removed (by unscrewing, snapping off or other method). This enables the inner swab to be removed, and the oral sample then analyzed by another system, such as by a reference lab.

FIG. 6A shows portions of the oral collection device from a different angle.

FIG. 7 shows further detail of how the inner (or secondary) swab containing a duplicate “B” sample of the original specimen may be extracted and tested further.

FIG. 8 shows an alternate view of the container (here the container is transparent), inner swab carriage, inner swab and associated end-cap, swab holder, and outer swab.

### DETAILED DESCRIPTION OF THE INVENTION

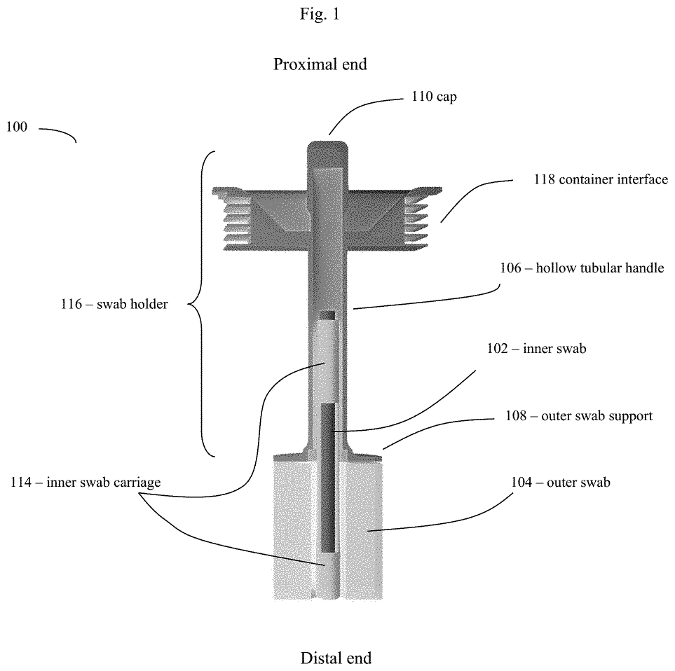

FIG. 1 shows a cross-sectional diagram of the oral collection device (100), showing the inner swab (102), outer swab (104), swab holder (116), and the inner swab carriage (114). The proximal end of the swab holder has a removable cap (110). The swab holder (116) also has an optional container interface (118), and a hollow tubular handle (106). The moveable inner swab carriage (114) holding an inner swab (102) is positioned inside of the hollow tubular handle (106). The distal end of the swab holder has an outer swab support (108), and a cylindrical outer swab (104). An opening along the central axis of the outer swab is positioned on the distal end of the outer swab support (108). Prior to use, the movable inner swab carriage (114) and the inner swab (102) are disposed inside this opening, as well as inside the hollow tubular handle (106).

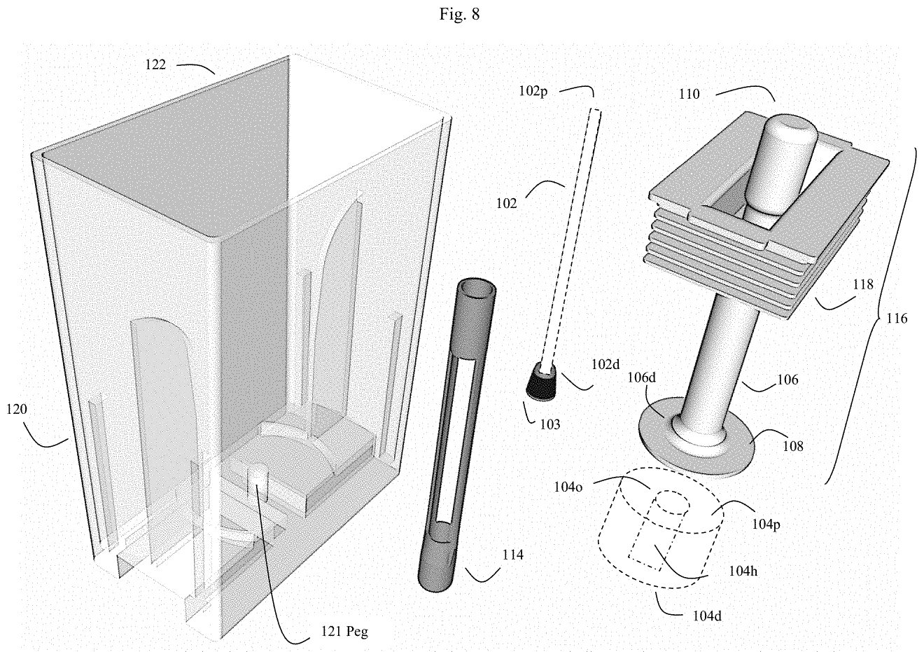

FIG. 8 shows an alternate view of the system components, further showing an optional container (120) (here the container is transparent) as well as the, inner swab carriage, (114) inner swab (102) and associated end-cap (110), swab holder (114), and outer swab (104).

Put alternatively, in some embodiments, the invention may be an oral fluid collection system. This system may comprise an oral collection device (100) with an inner swab (102), an outer swab (104), a swab holder (116) and an inner swab carriage (114). As shown in FIG. 1 and elsewhere, the swab holder can comprise a hollow tubular handle (106), an outer swab support (108), and a removable cap (110). In some embodiments, the swab holder may also comprise a container interface (118) configured to allow the swab holder to fit into an optional container (120.

The inner swab will typically comprise a substantially cylindrical first structure with a first height and first radius, an inner swab proximal end FIG. 8 (102p) and an inner swab distal end FIG. 8 (102d). In some embodiments, there also may be an end cap (103) configured at the distal end of the inner swab (102d).

The inner swab carriage (114) will typically be configured to hold at least the inner swab including the inner swab proximal end (102p) and the inner swab distal end (102d, and to move (at least upon the application of external force) along the central axis of the hollow tubular handle (106) while holding the inner swab.

The outer swab (104) will typically comprise a hollow and substantially cylindrical second structure with a second height and second radius, an outer swab proximal end (104p), and an outer swab distal end (104d). This outer swab (104) will typically further comprising a hollow cylindrical opening (104o) though the main axis of this substantially cylindrical second structure. This hollow cylindrical opening is typically configured to enable the inner swab carriage (114) and the inner swab (102) to fit inside this hollow opening.

Both the inner swab (102) and the outer swab (104) will typically comprise bibulous hydrophilic material configured to absorb saliva, and to release at least some of this saliva upon application of external pressure.

The swab holder (116) will typically be configured with the removable cap (100) on the swab holder proximal end, and the outer swab support (108) on the swab holder distal end. The removable cap (110) can be configured to reversibly mount to the swab holder proximal end. Further, the inner swab carriage (114) holding the inner swab (102) are configured to fit into a distal end of the hollow tubular handle (106).

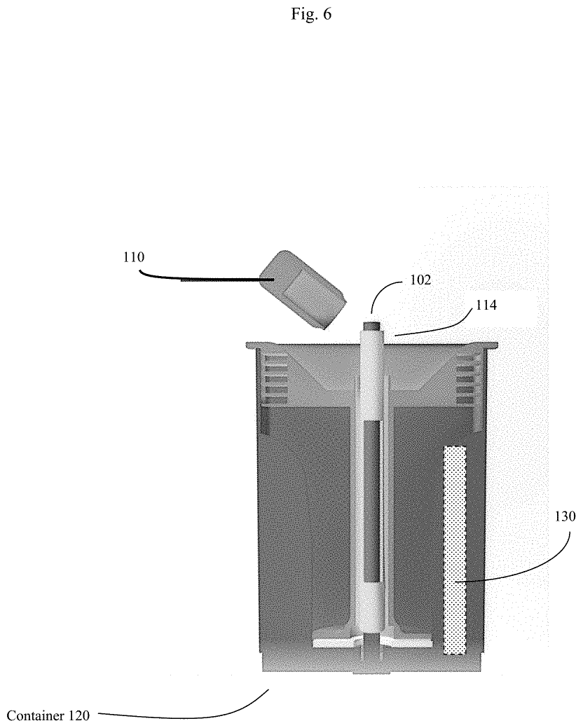

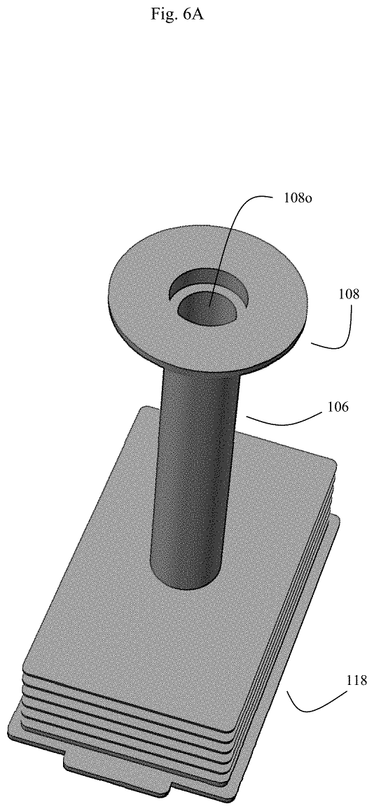

The distal end of the swab holder (116) will typically comprise the outer swab support (108). This outer swab support will typically comprise a disk-shaped annular structure comprising an annular hole (FIG. 6, 108o) through a center of the outer swab support (108). This annular hole will typically have a radius configured to allow the inner swab carriage (114) and the inner swab (102) to pass through the annular hole (108o) but to not allow the outer swab (104) to pass through the annular hole. So, the outer swab remains firmly in place upon application of external pressure.

The inner swab carriage is (114) is configured so that while holding the inner swab (102), the swab carriage can be disposed within the hollow tubular handle (106), pass through the annular hole (108o), and pass through the hollow cylindrical opening (104h) of the outer swab (104), thus creating an oral fluid collection system with a distal end comprising both the inner swab distal end (102d) and the outer swab distal end (104d), and a proximal end comprising the swab holder proximal end (116) and the removable cap (110).

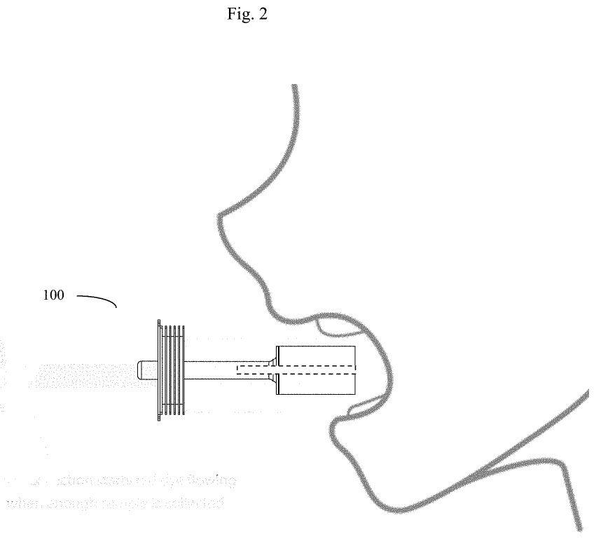

FIG. 2 shows the oral sample collection device (100) inserted into a patient's mouth in order to obtain a saliva sample.

As shown in FIG. 2 and FIG. 8, the system is configured to receive saliva from a donor's mouth, and store at least some of this saliva in at least portions of the inner swab (102) that are disposed within the hollow tubular handle (106), and to store at least some of this saliva in the outer swab (104) and portions of the inner swab (102) that are disposed within the hollow opening (104h) of the outer swab.

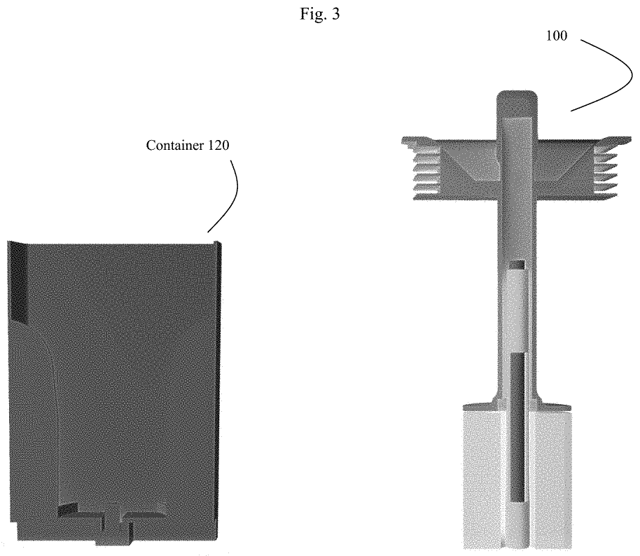

FIG. 3 shows a cross sectional view of both the oral collection device (oral fluid collection system 100) and the optional container (drug collection container) (120), which in this case is a drug screen container. Here, the oral fluid collector is placed into the donor's mouth (as shown in FIG. 2), and slid between the donor's lips and gums for 2-3 minutes. The movement helps both swabs to collect the saliva specimen.

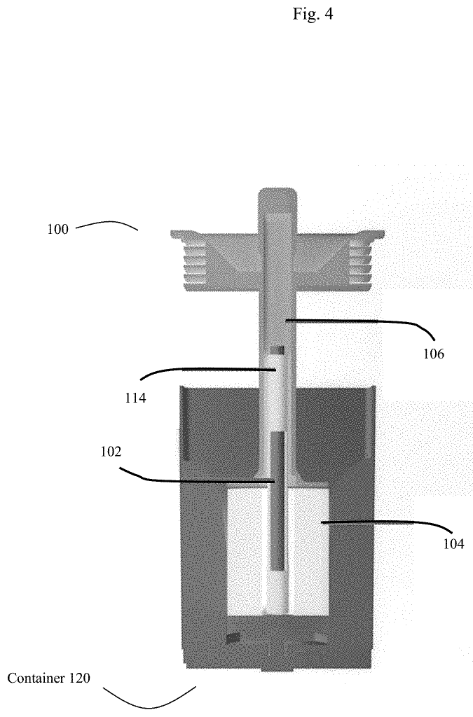

FIG. 4 shows how after the sample donor has applied saliva to the oral sample collection device, the collection device may be inserted into a container (120), such as container that may hold various drug screening point of care tests, or a container that can be used to transport material to a laboratory, or both. In this process, the oral sample (saliva) permeates both the outer swab and the inner swab.

As shown in FIG. 3 and FIG. 4, in some embodiments, the system may be configured so that so that application of external pressure (such as to cap 110, or container interface 118) can cause at least some of the saliva stored in the outer swab (104) (and optionally portions of the inner swab 102) to be released, while retaining other portions of the saliva stored in portions of the inner swab (102) that are disposed within the hollow tubular handle (106).

The outer swab (104) is a cylinder shape, with a hole down the center. The specimen collected in this outer swab is termed initial specimen “A”. The inner swab (102) collects a secondary or “B” specimen for testing at a later date

The inner swab cartridge (114) carries the inner swab (102) up into the swab holder's hollow tubular handle (106) when the oral fluid collector (100) is compressed. This inner swab (102) collects a secondary or “B” specimen for testing at a later date.

The swab holder (116) has a hollow tubular handle (106) which is airtight when fully closed, thus trapping a duplicate portion of the specimen inside (in inner swab 102).

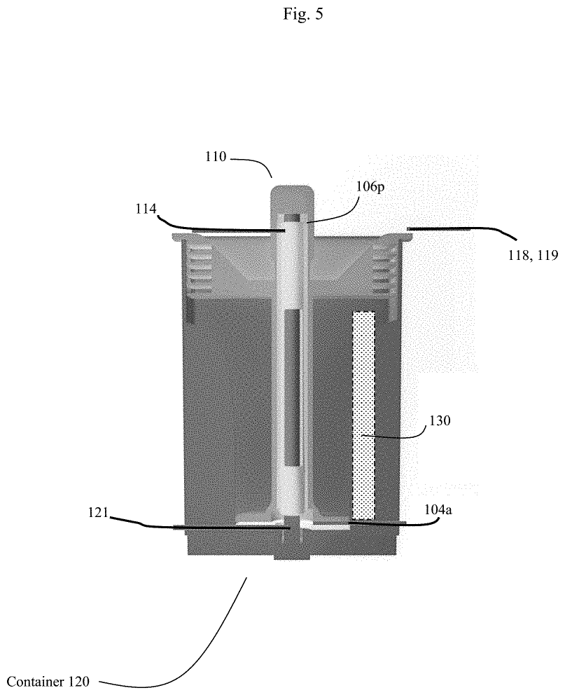

FIG. 5 shows the next step of the process. Here the collection device (100) is pressed down into the container (120) until the sample collector's container interface (118) is locked into position inside the container's open top. Here, the sample collector's container interface (118) can, for example, be locked using a one-way clip (119) on the top edge. This prevents the sample collector from coming loose, and also helps prevent sample tampering.

This pressing also causes the outer swab (104) to be compressed flat (104a). This releases the primary specimen “A” into the base of the container (120).

Put alternatively, the pressing compresses the outer swab (104), expelling saliva from the outer swab into the bottom of container. This also forces the inner swab carriage (114) and the inner swab (102) further towards the proximal end of the hollow tubular handle (106).

In a preferred embodiment, the inner swab carriage (114) is slightly shorter than the inner swab (102). Thus, a portion of the inner swab (102) will protrude beyond the inner swab carriage (114). This makes it easier to remove the inner swab (102) later. (This later inner swab extraction (102) step is usually done at a remote laboratory for purposes of running a duplicate or confirmatory second test. By contrast, the sample in the outer swab (104) is typically run locally for rapid/initial screening purposes.)

Note that in some embodiments, the container (120) may be transparent, and have one or more test devices (130), such as lateral flow immunoassays, arranged on the inner sides of the container. These optional assays (130) can be activated by the sample (expelled from outer swab 104), allowing one or more rapid tests (such as drugs of abuse tests) to be quickly performed. At the same time, a portion of the original sample is preserved in the inner swab (102), and this can optionally be tested at a later date. To do this, the container (120) and oral collection device assembly (100), which due to the latching action of the container interface (118) with the top of the container (FIG. 8, 122) may be preserved intact for further use.

In this embodiment, (see FIG. 1 and FIG. 8) the system further comprises a container (120). This container further comprises an outside, and inside, a bottom, and an open top (122). The swab holder (116) will further comprise a container interface (118) on or near the proximal end of the swab holder. In this embodiment, the container may be configured with an open top (122) complementary to the container interface (118), as well as a hollow interior configured to enable the device (100) (e.g., the swab holder (116), outer swab (104), the inner swab carriage 114) to be inserted into this hollow interior, and to enable the container interface (118) to form a seal with at least a portion of the inside of the open top (122).

Note that in some embodiments, the base of the container (drug collection container 120) may also have a fixed peg (121) that acts to push the inner or secondary swab (102) into the swab holder's hollow tubular handle (106). This tubular handle is often made of air impermeable materials such as plastic, and either automatically or manually may then be further rendered air-tight afterwards to help preserve the secondary sample in inner swab (102).

FIG. 6 shows how the duplicate sample preserved in the inner swab (102) may be accessed. Here the removable cap (110) may be removed (by unscrewing, snapping off or other method) and discarded if desired. This enables the inner swab (102) to be removed, and the secondary “B” oral sample specimen then analyzed by another system, such as by a reference lab.

FIG. 6A shows portions of the oral collection device from a different angle.

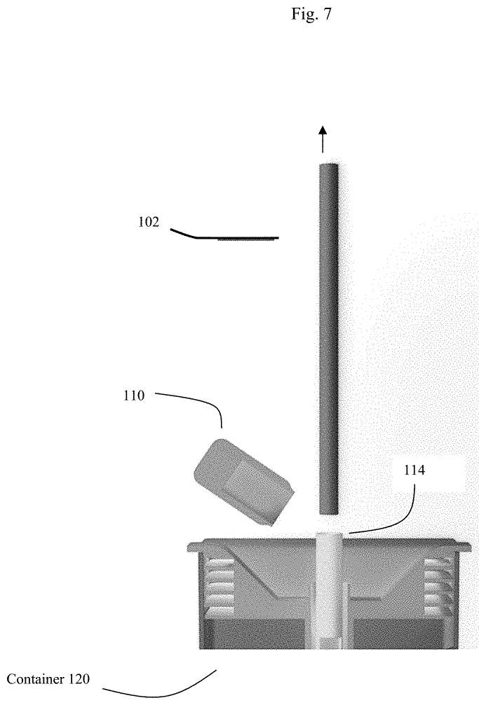

FIG. 7 shows further detail of how the inner (or secondary) swab (102) containing a duplicate “B” sample of the original specimen may be extracted and tested further.

As shown above, in some embodiments, the system may be configured so that after the cap (110) is removed, the inner swab (102) (either with or without the inner swab carriage) is configured be removable from the hollow tubular handle (106). The internal swab (102) is configured so that additional portions of the saliva (the “B” sample) stored in the portions of the inner swab that were previously disposed inside the hollow tubular handle (106) can be expelled by application of external pressure.

As shown in FIG. 5 and FIG. 6, in some embodiments, the container (120) can be further configured to hold at least one lateral flow immunoassay (130) or other rapid test. The bottom of the lateral flow immunoassay is typically further configured so that released saliva (now on the bottom of container 120) can be analyzed by the at least one lateral flow immunoassay. Often, the sides of the container (120) may be transparent, so that the results of the immunoassays (s) can be directly observed through the transparent walls of the container.

Although often the container may contain one or more rapid assays, this is not an absolute requirement. In some embodiments, the container may not contain any assays. In this case, the container may be further configured so that released saliva from the outer swab (104) can be retrieved by a pipette or other fluid sampling system, and then tested elsewhere

### Further Discussion

In these examples, the dimensions are approximate. Assume that all stated dimensions can vary by plus or minus 50%.

Referring to FIG. 1, and FIG. 8, in some embodiments, the inner swab material (102) may be a cylinder with a height of approximately 90 mm and a radius of approximately 3.5 mm. The outer swab (104) may be a hollow cylinder with a height of about 30 mm, an outer radius of approximately 15 mm, with a hollow cylindrical hole of approximately 3.5 mm (or otherwise substantially similar to the radius of the inner swab) through the main axis of the outer swab.

Put alternatively, as shown in FIG. 1, the oral fluid collection system may comprise an inner swab (102), an outer swab (104), an inner swab carriage (114), and a swab holder (116). This swab holder can comprise an optional container interface (118), a hollow tubular handle (swab handle) (106), an outer swab support (108), and a removable cap (110).

As shown in FIG. 8, the inner swab (102) will typically comprise a substantially cylindrical first structure with a first height and first radius, an inner swab proximal end (102p) and an inner swab distal end (102d). The outer swab will typically comprise a hollow and substantially cylindrical second structure with a second height and second radius, an outer swab proximal end (104p), and an outer swab distal end (104d). This inner swab (102) is typically mounted inside the opening of an inner swab carriage (114). This inner swab carriage allows the inner swab to move up and down along the axis of the hollow tubular handle (in response to force) without causing the inner swab (102) to fold or buckle. The inner swab carriage (114) can also reduce friction that might otherwise impede movement of the inner swab (102).

The outer swab (104) will also typically further comprise a hollow cylindrical opening (104o) though a main axis of this substantially cylindrical second structure. This hollow cylindrical opening is typically configured to enable the inner swab (102) and the inner swab carriage (114) to fit inside this hollow opening (104o).

As will be discussed shortly, the inner swab and the outer swab will typically comprise a bibulous hydrophilic material configured to absorb saliva and to release at least some of this saliva upon application of external pressure.

As shown in FIG. 1 and FIG. 8, the swab holder portion (116) of the oral fluid collection system (100) will also comprise a hollow tubular handle (106) comprising a handle proximal end (106p) and a handle distal end (106p). The removable cap (110) is typically configured to reversibly mount to the handle proximal end (106p). This can be by a screw thread, or bayonet and snap joint, or other mechanism. Further, the inner swab proximal end (102p) is typically configured to fit into inner swab carriage (114), and the resulting combination fits into the hollow tubular handle distal end (106d) of the hollow tubular handle, and often extended all or nearly all the way in the hollow handle to about the level of the handle proximal end (106p).

The swab holder (116) portion of the oral fluid collection system will also typically comprise an outer swab support (108). This outer swab support (108) typically comprises a disk-shaped annular structure comprising an outer swab support hole (see FIG. 6A, 108o) through the center of the outer swab support (108). This outer swab support hole (108o) will typically have a radius configured to allow just the inner swab carriage (114) and the inner swab (102) to pass through the outer swab support hole, and not allow either the handle distal end (106d) of the hollow tubular handle (106) or the outer swab (104, 104p) to pass through the swab hole (FIG. 6A, 108o). In essence, the outer swab support (108) acts to “mount” the outer swab on the swab holder, with the inner swab (102) and inner swab carriage (114) providing at least some additional stabilizing force to keep the outer swab (104) so mounted.

Thus, the inner swab (102) is configured to be at least partially disposed within both the inner swab carriage (114) and the hollow tubular handle (106), pass through the swab holder hole (108o), and pass through the outer swab (104) and swab hole (104o), thus creating an oral fluid collection system with a distal end comprising both the inner swab distal end (102d) and the outer swab distal end (104d). The collection system also has a proximal end comprising the removable cap (110) mounted onto hollow tubular handle proximal end (FIG. 5, 106p)

As previously discussed, both the inner and outer swab material will typically comprise a bibulous hydrophilic material, configured to readily absorb the saliva sample by wicking or capillary action, hold the sample, and then release the sample either by direct contact with another bibulous hydrophilic material (e.g., such as upon contact between the outer swab and the inner swab) or upon application of external pressure. Suitable materials may comprise synthetic sponge or foam, cotton, synthetic fibers, preferably hydrophilic fibers), and the like.

In some embodiments, the hollow tubular handle (106) may be a hollow tube, preferably a hollow cylindrical plastic tube made from a transparent or translucent plastic, with a height of approximately 60 mm, outer radius of approximately 8 mm, and a hollow cylindrical interior along the main axis of the plastic tube with an inner radius about 6 mm, slightly larger than the radius of the inner swab carriage (114).

As shown in FIG. 2, the system is typically configured to receive saliva from a donor's mouth, and store at least some of this saliva in at least portions of the inner swab (102), such as the proximal portions that are disposed within the hollow tubular handle (106), and to store at least some the saliva in both the outer swab (104) and at least the distal portions of the inner swab (102) that are disposed within the outer swab (104).

### Optional Preservative Solution:

In some embodiments, it may be useful to further supply a liquid preservative solution (132). This preservative solution may be based on either an aqueous buffer, or an organic fluid such as an alcohol, or mixtures thereof. This optional liquid preservative solution may contain anti-bacterial agents (used to protect the sample from bacterial degradation during storage or transport), or other agents useful to preserve the integrity of the saliva sample over a period of time, often up to several days or even a week.

### Drugs of Use Testing Example.

In the following example, assume that the oral sampling device is being used to first deliver a first portion of an oral saliva sample to a local dry reagent drugs of abuse test, such as the commonly owned and invented system previously disclosed in U.S. provisional patent 62/546,135; US patent publication 2019/0057759 (published Feb. 21, 2019); and U.S. Pat. No. 10,340,032 (issued Jul. 2, 2019). The entire contents of U.S. provisional patent 62/546,135, US patent publication 2019/0057759; and U.S. Pat. No. 10,340,032 are incorporated herein by reference. The oral sampling device will then be used to deliver a second portion of the same oral sample to a remote reference laboratory for confirmation and/or additional analysis.

In this example, assume that there is a donor supplying sample, and an operator who both obtains the sample and then runs a first portion of the sample on a local dry reagent test, such as a lateral flow immunoassay test, according to the methods discussed in the above patent applications and patents. There will also be a remote laboratory with a second operator that processes the remaining part of the oral sample.

The operator inserts the distal end of the swab oral sampling device (e.g., the end with the first and second swab) into the container (120), which may often further contain one or more rapid drugs of abuse tests, such as lateral flow immunoassays.

This container may have a fixture configured to hold the oral sampling device and to conduct sample fluid expelled from the oral sampling device into the actual test portion of the rapid drug test. To facilitate this process, the operator may put pressure on the container interface (118). This pressure will act to partially compress the distal end of both the outer swab (104d) and the embedded inner swab (102d), compressing the distal ends of both swabs, and expelling a portion of the sample fluid into the rapid drug test device. This is shown in FIG. 5.

Put alternatively, in some embodiments, the system may be configured so that application of external pressure (on cap 110, container interface 118, and hollow tubular handle 106) can act much like a plunger. This causes at least some of the saliva stored in the outer swab (104) to be released. This action also pushes the inner swab carriage (114) and the inner swab (102) further up into the hollow tubular handle (106). As a result, at least some other “B” portions of the saliva sample remain stored in the portions of the inner swab (102) disposed within the hollow tubular handle (106).

Note further than in some embodiments, after cap (110) is removed, the inner swab (102) can be removed from the hollow tubular handle (106). Here additional portions of the saliva that were stored in the inner swab that were previously disposed inside the hollow tubular handle (106) can then be expelled at the second lab by application of external pressure.

In some embodiments, if the rapid test kit (such as FIG. 5 and FIG. 6 130) doing the first test indicates a negative result, the entire device can then be discarded. However, if the first (rapid) test indicates a positive result (e.g., the donor is testing positive for one or more particular drugs of abuse), further confirmation testing on a different portion of this sample (the “B” sample stored in 102) is needed. To do this, the entire device may be shipped to a second laboratory for further confirmation.

### At the Second (Confirmation/Verification/Reference) Lab.

When the transport/confirmation tube arrives at the second lab, operators at the second lab will unscrew or otherwise remove the swab cap (110) the device. This exposes the proximal end of the inner swab (102p), and gives the second lab the ability to remove this inner swab material (102).

The second lab may then process the remaining portion of the sample as desired for further testing.

1. An oral fluid collection system comprising:
An inner swab, an outer swab, a swab holder, an inner swab carriage;
said swab holder comprising a hollow tubular handle;
said inner swab carriage configured to hold at least some of said inner swab, and to move along an axis of said hollow tubular handle while holding said inner swab;
said outer swab comprising a hollow and substantially cylindrical second structure with a second height and second radius, an outer swab proximal end, and an outer swab distal end;
said outer swab comprising a hollow cylindrical opening though a main axis of said substantially cylindrical second structure, said hollow cylindrical opening configured to enable said inner swab carriage and said inner swab to fit inside said hollow opening;
said inner swab and said outer swab comprising bibulous hydrophilic material configured to absorb saliva, and to release at least some of said saliva upon application of external pressure;
said inner swab carriage configured, while holding said inner swab, to be disposed within said hollow tubular handle, thus creating an oral fluid collection system with a distal end comprising both an inner swab distal end and said outer swab distal end, and a proximal end comprising a swab holder proximal end.

2. The system of claim 1, further configured to receive saliva from a donor's mouth, and store at least some of said saliva in at least portions of said inner swab that are disposed within said hollow tubular handle, and to store at least some said saliva in said outer swab and portions of said inner swab that are disposed within said outer swab.

3. The system of claim 1, further configured so that application of external pressure causes at least some of said saliva stored in said outer swab to be released, while retaining other portions of said saliva stored in portions of said inner swab disposed within said hollow tubular handle.

4. The system of claim 3, further configured so that said inner swab is configured be removable from said hollow tubular handle, and portions of said saliva stored in the portions of said inner swab that were previously disposed inside said hollow tubular handle can be expelled by application of external pressure.

5. The system of claim 3, further comprising a container;
said container comprising an outside, and inside, a bottom, and an open top;
said swab holder further comprising a container interface on said swab holder proximal end;
said container configured with an open top complementary to said container interface, and a hollow interior configured to enable said swab holder, said outer swab, said inner swab carriage to be inserted into said hollow interior, and to enable said container interface to form a seal with at least a portion of said inside of said open top.

6. The system of claim 5, wherein said container is further configured to hold at least one lateral flow immunoassay, said bottom and said lateral flow immunoassay further configured so that released saliva can be analyzed by said at least one lateral flow immunoassay.

7. The system of claim 5, wherein said container is further configured so that released saliva can be retrieved by a pipette or other fluid sampling system.

8. The system of claim 1, wherein said swab holder further comprises an outer swab support and a removable cap.

9. An oral fluid collection system comprising:
An inner swab, an outer swab, a swab holder, an inner swab carriage;
said swab holder comprising a hollow tubular handle, outer swab support, and a removable cap;
said inner swab comprising a substantially cylindrical first structure with a first height and first radius, an inner swab proximal end and an inner swab distal end;
said inner swab carriage configured to hold at least said inner swab proximal end and said inner swab distal end, and to move along an axis of said hollow tubular handle while holding said inner swab;
said outer swab comprising a hollow and substantially cylindrical second structure with a second height and second radius, an outer swab proximal end, and an outer swab distal end;
said outer swab further comprising a hollow cylindrical opening though a main axis of said substantially cylindrical second structure, said hollow cylindrical opening configured to enable said inner swab carriage and said inner swab to fit inside said hollow opening;
said inner swab and said outer swab comprising bibulous hydrophilic material configured to absorb saliva, and to release at least some of said saliva upon application of external pressure;
said swab holder configured with said removable cap on a swab holder proximal end, and said outer swab support on a swab holder distal end;
said removable cap configured to reversibly mount to said swab holder proximal end;
said inner swab carriage and said inner swab proximal end configured to fit into a distal end of said hollow tubular handle;
said distal end of said swab holder comprising said outer swab support, said outer swab support comprising a disk-shaped annular structure comprising an annular hole through a center of said outer swab support, said annular hole having a radius configured to allow said inner swab carriage and said inner swab to pass through said annular hole, but to not allow said outer swab to pass through said annular hole;
said inner swab carriage configured, while holding said inner swab, to be disposed within said hollow tubular handle, pass through said annular hole, and pass through said hollow cylindrical opening of said outer swab, thus creating an oral fluid collection system with a distal end comprising both said inner swab distal end and said outer swab distal end, and a proximal end comprising said swab holder proximal end and said removable cap.

10. The system of claim 9, further configured to receive saliva from a donor's mouth, and store at least some of said saliva in at least portions of said inner swab that are disposed within said hollow tubular handle, and to store at least some said saliva in said outer swab and portions of said inner swab that are disposed within said outer swab.

11. The system of claim 9, further configured so that application of external pressure causes at least some of said saliva stored in said outer swab to be released, while retaining other portions of said saliva stored in portions of said inner swab disposed within said hollow tubular handle.

12. The system of claim 11, further configured so that after said outer swab and said cap are removed, said inner swab is configured be removable from said hollow tubular handle, and said inner swab is configured so that additional portions of said saliva stored in portions of said inner swab that were previously disposed inside said hollow tubular handle can be expelled by application of external pressure.

13. The system of claim 11, further comprising a container;
said container comprising an outside, and inside, a bottom, and an open top;
said swab holder further comprising a container interface on said swab holder proximal end;
said container configured with an open top complementary to said container interface, and a hollow interior configured to enable said swab holder, said outer swab, said inner swab carriage to be inserted into said hollow interior, and to enable said container interface to form a seal with at least a portion of said inside of said open top.

14. The system of claim 13, wherein said container is further configured to hold at least one lateral flow immunoassay, said bottom and said lateral flow immunoassay further configured so that released saliva can be analyzed by said at least one lateral flow immunoassay.

15. The system of claim 13, wherein said container is further configured so that released saliva can be retrieved by a pipette or other fluid sampling system.

16. A method of collecting oral fluids for testing, said method comprising:
using an oral fluid collection system to obtain saliva from a patient, said oral fluid collection system comprising at least an outer swab and an inner swab, both said swabs mounted on a swab holder, and said inner swab is mounted on an inner swab carriage positioned inside said swab holder;
said both said swabs comprising a bibulous hydrophilic material;
collecting saliva on both swabs simultaneously;
testing saliva retained on said outer swab; and
storing said inner swab for later testing.

17. The method of claim 16, wherein said oral fluid collection system further comprises:
said inner swab, said outer swab, said swab holder, and said inner swab carriage;
said swab holder comprising a hollow tubular handle;
said inner swab carriage configured to hold at least some of said inner swab, and to move along an axis of said hollow tubular handle while holding said inner swab;
said outer swab comprising a hollow and substantially cylindrical second structure with a second height and second radius, an outer swab proximal end, and an outer swab distal end;
said outer swab comprising a hollow cylindrical opening though a main axis of said substantially cylindrical second structure, said hollow cylindrical opening configured to enable said inner swab carriage and said inner swab to fit inside said hollow opening;
said inner swab and said outer swab comprising bibulous hydrophilic material configured to absorb saliva, and to release at least some of said saliva upon application of external pressure;
said inner swab carriage configured, while holding said inner swab, to be disposed within said hollow tubular handle, thus creating an oral fluid collection system with a distal end comprising both an inner swab distal end and said outer swab distal end, and a proximal end comprising said swab holder proximal end.

18. The method of claim 16, wherein said oral fluid collection system further comprises:
said inner swab, said outer swab, said swab holder, and said inner swab carriage;
said swab holder comprising a hollow tubular handle, outer swab support, and a removable cap;
said inner swab comprising a substantially cylindrical first structure with a first height and first radius, an inner swab proximal end and an inner swab distal end;
said inner swab carriage configured to hold at least said inner swab proximal end and said inner swab distal end, and to move along an axis of said hollow tubular handle while holding said inner swab;
said outer swab comprising a hollow and substantially cylindrical second structure with a second height and second radius, an outer swab proximal end, and an outer swab distal end;
said outer swab further comprising a hollow cylindrical opening though a main axis of said substantially cylindrical second structure, said hollow cylindrical opening configured to enable said inner swab carriage and said inner swab to fit inside said hollow opening;
said inner swab and said outer swab comprising bibulous hydrophilic material configured to absorb saliva, and to release at least some of said saliva upon application of external pressure;
said swab holder configured with said removable cap on swab holder proximal end, and said outer swab support on said swab holder distal end;
said removable cap configured to reversibly mount to said swab holder proximal end;
said inner swab carriage and said inner swab proximal end configured to fit into a distal end of said hollow tubular handle;
said distal end of said swab holder comprising said outer swab support, said outer swab support comprising a disk-shaped annular structure comprising an annular hole through a center of said outer swab support, said annular hole having a radius configured to allow said inner swab carriage and said inner swab to pass through said annular hole, but to not allow said outer swab to pass through said annular hole;
said inner swab carriage configured, while holding said inner swab, to be disposed within said hollow tubular handle, pass through said annular hole, and pass through said hollow cylindrical opening of said outer swab, thus creating an oral fluid collection system with a distal end comprising both said inner swab distal end and said outer swab distal end, and a proximal end comprising said swab holder proximal end and said removable cap.
